(19) United States
(12) Patent Application Publication
(10) Pub. No.: US 2004/0152211 A1
(43) Pub. Date: Aug. 5, 2004

(54) System and method for multiplexed biomolecular analysis

(75) Inventors: Arun Majumdar, Orinda, CA (US); Min Yue, Albany, CA (US); Chris Dames, Brighton, MA (US); Richard Cote, Los Angeles, CA (US); Henry Lin, San Marino, CA (US); Ram Datar, Alhambra, CA (US); Srinath Satyanarayana, Berkeley, CA (US)

Correspondence Address: BURNS DOANE SWECKER & MATHIS L L P, POST OFFICE BOX 1404, ALEXANDRIA, VA, 22313-1404, US

(73) Assignee: The Regents of the University of California, Oakland, CA

(21) Appl. No.: 10/715,017

(22) Filed: Nov. 17, 2003

Related U.S. Application Data
(60) Provisional application No. 60/426,851, filed on Nov. 15, 2002.

(51) Int. Cl. G01N 33/543

(52) U.S. Cl. 436/518; 435/6

(57) ABSTRACT

Systems and techniques for biomechanical analysis are described. Multiple sensors are used to detect different molecules and/or to analyze multiple samples. A property of the sensors changes in response to a target molecule. The change in the property is detected, which may provide a measure of the concentration of the target molecule.

### RELATED APPLICATION

This application claims priority under 35 U.S.C. § 119 to U.S. Provisional Application No. 60/426,851 entitled Multiplexed Biomolecular Analysis, filed on Nov. 15, 2002, the entire disclosure of which is incorporated herein by reference in its entirety for all purposes.

### GOVERNMENT INTEREST

The present invention was made with Government support under Grant (Contract) No. R21CA86132-01 awarded by the National Institutes of Health/National Cancer Institute and Contract No. DE-FG03-98ER14870 awarded by the United States Department of Energy. The Government has certain rights to this invention.

### TECHNICAL FIELD

The present invention relates to micro- structures and nano-structures for molecular analysis.

### BACKGROUND OF THE INVENTION

In order to identify particular biological molecules, a probe molecule may be used, where the probe molecule interacts with the particular biological molecule to be detected. For example, in order to detect particular DNA material, a short single-stranded DNA (ssDNA) sequence may be used as a probe molecule for a complimentary ssDNA. Similarly, in order to detect a particular antigen, an appropriate antibody may used as a probe molecule.

The presence of the particular biological molecule may be detected by functionalizing a surface using an appropriate probe molecule, and then detecting a physical change in the functionalized area. For example, probe molecules may be attached to a surface of a cantilever. When the molecule to be detected binds to one of the probe molecules on the surface of the cantilever, the cantilever may bend due to a change in the surface stress. Determining the amount by which the cantilever bends may provide a measure of the concentration of the molecule to be detected.

### SUMMARY OF THE INVENTION

The present invention provides systems and techniques for detecting different molecules (including biomolecules) using an array of sensors. In preferred embodiments, the sensors may be micromechanical surface stress sensors (MSSS), such as microcantilevers and membranes. Preferably, such sensors change in response to one or more molecular or biomolecular interactions.

In accordance with the present invention, current techniques (including DNA hybridization, antigen-antibody binding, protein-protein interactions, and DNA-protein interactions) may be used for high-throughput biomolecular analysis.

### BRIEF DESCRIPTION OF THE DRAWINGS

FIG. 1A is an illustration of a sensor array incorporating the present invention.

FIG. 1B is a top plan view of a cantilever according to an embodiment of the present invention.

FIG. 1C is an enlarged view of a portion of the cantilever of FIG. 1B.

FIG. 1D is a side elevation view of a bending microcantilever.

FIG. 1E is a perspective view of an optional embodiment of a preferred cantilever.

FIG. 1F is a side elevation view of another optional embodiment of a preferred cantilever.

FIG. 1G is a sectional side elevation view of a microfluidic chamber incorporating a microcantilever in accordance with the present invention.

FIG. 2A is an illustration of a cantilever prior to target molecule binding thereto.

FIG. 2B is an illustration of a cantilever after target molecule binding thereto.

FIG. 3A is a sectional side elevation view of a first step in fabricating a cantilever in accordance with the present invention.

FIG. 3B is a sectional side elevation view of a second step in fabricating a cantilever in accordance with the present invention.

FIG. 3C is a sectional side elevation view of a third step in fabricating a cantilever in accordance with the present invention.

FIG. 3D is a sectional side elevation view of a fourth step in fabricating a cantilever in accordance with the present invention.

FIG. 3E is a sectional side elevation view of a fifth step in fabricating a cantilever in accordance with the present invention.

FIG. 3F is a top plan view corresponding to FIG. 3E, further showing a fluid or gas through-hole below the cantilever.

FIG. 3G is a perspective view corresponding to FIG. 3F.

FIG. 4A is a top plan view of a microfluidic system schematically showing different sensors being functionalized to detect different target molecules.

FIG. 4B is a top plan view of a microfluidic system schematically showing different sensors concurrently being exposed to the same fluid or gas for detection of different target molecules therein.

FIG. 4C is an enlarged perspective view of first embodiment of a microfluidic cell for use in the system of FIGS. 4A/4B.

FIG. 4D is an enlarged perspective view of second embodiment of a microfluidic cell for use in the system of FIGS. 4A/4B.

FIG. 5A is a schematic illustration of the operation of an optical interferometry detection system in accordance with the present invention.

FIG. 5B another schematic illustration of the operation of an optical interferometry detection system in accordance with the present invention.

FIG. 5C illustrates the relationship between fringe intensity and cantilever detection on a CCD screen.

FIG. 5D is an illustration of an array of interference patterns on a CCD screen.

FIG. 5E is a perspective view of an array of cantilever sensors in accordance with the present invention.

FIG. 6A is a is a schematic illustration of the operation of a ray-based optical detection system in accordance with the present invention.

FIG. 6B another schematic illustration of the operation of a ray-based optical detection system in accordance with the present invention.

FIG. 6C illustrates the difference in position between two spots on the image screen of FIG. 6B.

FIG. 7 is a schematic illustration of a system arrangement in accordance with the present invention.

FIG. 8 is a 1-D representation of a beam spot intensity profile measured by a CCD array.

FIG. 9 is an illustration of spot movement on a CCD image corresponding to temperature induced cantilever movement.

FIG. 10 is an enlarged view of regions of FIG. 10 illustrating a representative calculation of spot center of intensity performed by an image processing algorithm in accordance with the present invention.

FIG. 11A illustrates calculated temperature sensitivity of a spot center of intensity performed by an image processing algorithm in accordance with the present invention.

FIG. 11B illustrates calculated temperature sensitivity of a spot center of intensity performed by an image processing algorithm after assumption of a constant drift rate in accordance with the present invention.

FIGS. 12 to 16 are a series of images illustrating various stages in calculating gain factors, wherein:

FIG. 12 Pcal_i=Initial image for ΔP calibration

FIG. 13 Pcal_f=Final image for ΔP calibration

FIG. 14 Pcal=average of initial and final images

FIG. 15

Delta=initial−final.

FIG. 16

Gain factor matrix=Delta./Pcal.

FIG. 17 is a comparison of center of mass and gain factor algorithms.

FIG. 18 is a CCD image showing reflection spots of four different cantilevers.

FIG. 19 is a displacement vs. time plot showing movement of two different sensors, and relative movement therebetween.

FIG. 20 is a flow chart of calculation of a gain factor in accordance with the present invention.

FIG. 21 is pseudocode of calculation of a gain factor in accordance with the present invention.

FIG. 22 is an illustration of cantilever deflection responses as a function of time for different concentrations of an antigen.

FIG. 23 illustrates surface stresses for three different cantilever geometries caused by a particular binding reaction, showing that the binding reaction is independent of cantilever geometry.

FIG. 24 is a perspective view of a bank of cantilever sensors.

FIG. 25 illustrates Z-axis deflection along the X-axis of a cantilever showing the results of stress balancing of the cantilever.

FIG. 26 illustrates tip deflection of a cantilever as a function of temperature.

FIG. 27 illustrates calculated drift of a control spot in domain D2.

### DETAILED DESCRIPTION OF THE DRAWINGS

(1) Overview:

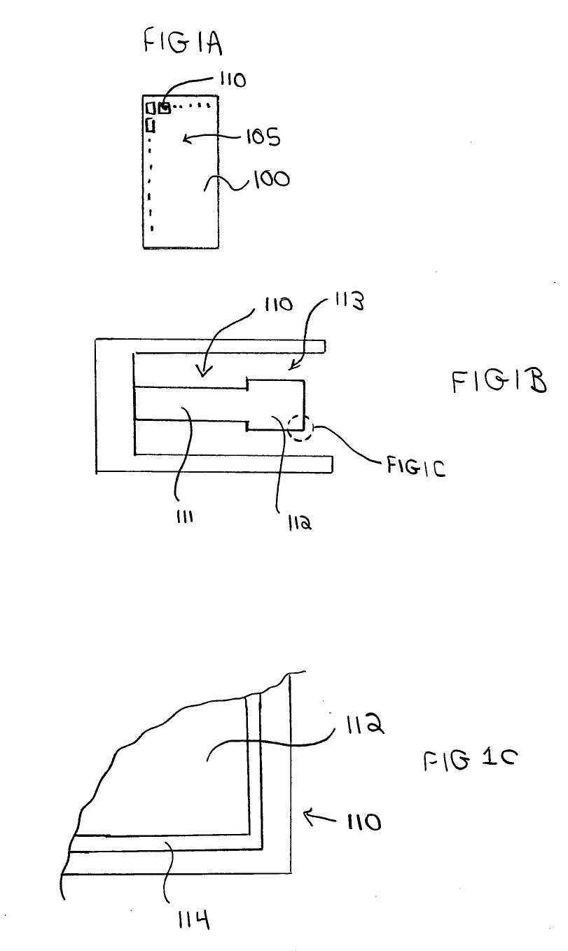

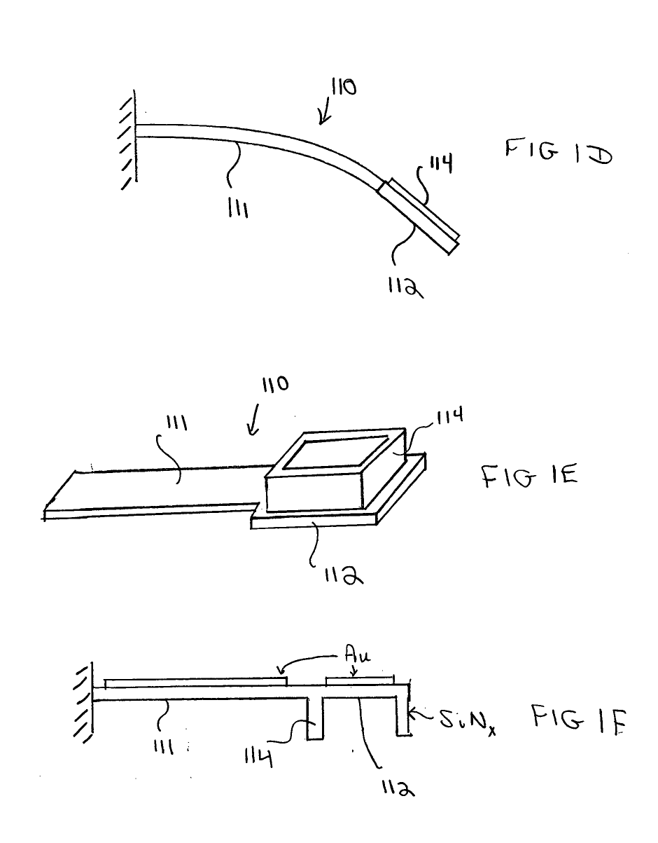

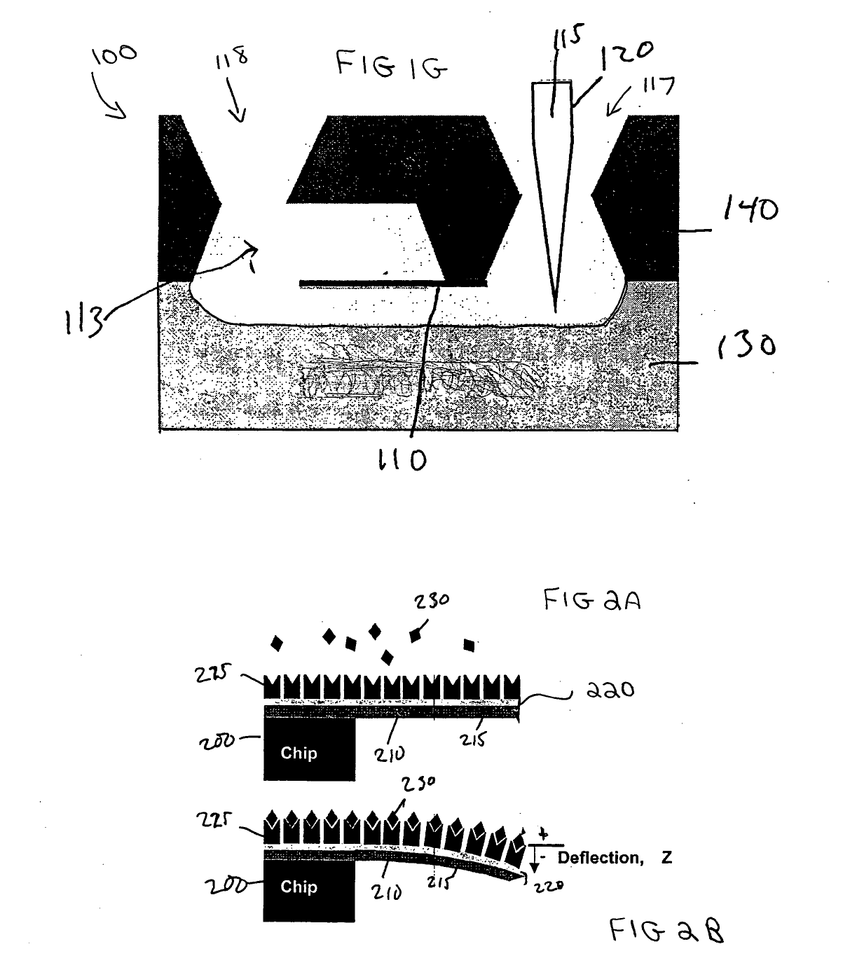

Referring to FIGS. 1A-1D, a chip 100 includes a sensor array 105. Array 105 includes an N×M (here, 6×15) array of MSSS such as cantilevers 110. FIG. 1B shows a top view of cantilever 110, while FIG. 1C shows a magnified view of a portion of cantilever 110. Cantilever 110 comprises a beam 111 and a paddle 112, where paddle 112 is generally reflective to provide an optical signal to be detected. As shown in FIG. 1C, a strengthening ridge 114 may optionally be provided. Preferably, ridge 114 runs around at least a portion of the perimeter of paddle 112. In various embodiments, ridge 114 may be positioned either on the top, or bottom, or both, of paddle 112. Ridge 114 operates to prevent paddle 112 from bending while beam 111 bends, as shown in FIG. 1D. Optionally, ridge 114 may form a “box” around paddle 112 as shown in FIG. 1D, or may comprise bottom features of paddle 112 as shown in FIG. 1E (wherein the bottom portion of cantilever 110 is made of SiNx and the top portion is made of gold).

Each cantilever 110 is associated with a reservoir 113 that may hold fluids, where the fluids may include fluids for functionalizing the cantilever, sample fluids for molecular or biological analysis, and fluids used for rinsing. Reservoir 113 may be formed in a silicon portion 140 of chip 100, a glass/polydimethylsiloxane (PDMS) portion 130 of chip 100, or both (as shown in the optional preferred embodiment of FIG. 1G). Reservoir 113 also provides a space into which cantilevers 110 can deflect.

Referring to FIG. 1G, a fluid 115 is introduced into reservoir 113 through inlet 117 using a device such as a micropipette 120. An outlet 118 is also provided. As can be seen, an advantage of the microfluidic system of FIG. 1G is that cantilever 110 may be completely submerged within fluid filing reservoir 113. Specifically, having an outlet 118 permits removal of any air bubbles adjacent to cantilever 110. As will also be explained, motion of cantilever 110 can be detected by reflecting laser light off cantilever 110 (with the laser light passing through clear portion 130 of chip 100. In preferred aspects, multiple micropipettes 120 may be used to functionalize multiple cantilevers 110 in array 105. Using this system, individual cantilevers 110 may be separately functionalized. (i.e. they may optionally be functionalized to detect different target molecules). Providing individual functionalization of cantilevers, as well as a technique for multiplexed measurement of cantilever deflection (see below), allows multiple molecules to be detected at the same time. Some cantilevers 110 may remain unfunctionalized to be used for common mode rejection (see below).

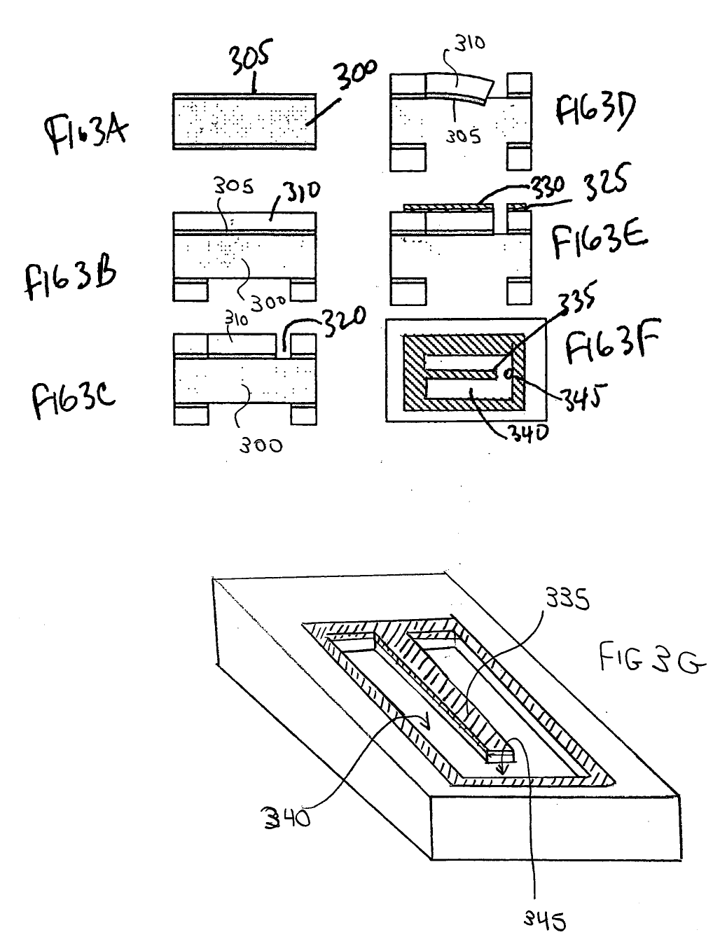

Referring to FIGS. 2A and 2B, a chip 200 includes multiple sensors such as a cantilever 210. Cantilever 210 includes a silicon nitride portion 215, and a gold portion 220. Cantilever 210 may include a chrome portion (not shown) between silicon nitride portion 215 and gold portion 220. Cantilever 210 includes probe molecules 225, which may be attached to a surface of cantilever 210 using, e.g, an intermediate thiol or cysteamine group. Probe molecules 225 may interact with particular target molecules 230.

The sensors exhibit a physical change in response to a biological interaction between probe molecules and target molecules. For example, when a target molecule 230 binds to a probe molecule 225, the surface stress of cantilever 210 changes, and cantilever 210 deflects in the -z direction. The amount of deflection provides a measure of the concentration of target molecules in a fluid.

Referring to FIGS. 3A-3F, a method for fabricating a sensor array is illustrated. FIGS. 3A-3F show formation of a single cantilever 110 (with reflecting paddle 112 removed for illustrative purposes).

Referring to FIG. 3A, a substrate such as a silicon substrate 300 is provided. Substrate 300 may be a single polished silicon <100> wafer. A thin, high-stress silicon nitride (SiNₓ) layer 305 is formed on substrate 300. For example, layer 305 may be formed in a low pressure chemical vapor deposition (LPCVD) furnace. Referring to FIG. 3B, a thicker, lower-stress SiNₓ layer 310 is formed on layer 305. Referring to FIG. 3C, an opening is patterned on the backside to act as a mask for an etch, and an opening 320 defining the cantilever is patterned on the frontside.

Referring to FIG. 3D, material from substrate 300 is etched to release the cantilever and to create a reservoir space proximate to the cantilever. The etch may be a wet potassium hydroxide (KOH) etch, or a wet tetramethyl ammonium hydroxide (TMAH) etch. Because of the stress in the nitride underlayer, the released cantilevers are generally bent downwards by many tens of microns at this stage.

Referring to FIG. 3E, a chrome adhesion layer 325 is formed on layer 320. A gold layer 330 is formed on layer 325. Residual stresses in the gold and chromium may pull the cantilever back close to neutral.

Referring to FIGS. 3F and 3G, a top view of the area of the array shows the cantilever 335 and reservoir 340. A through hole 345 may be etched through substrate 300 to provide a path for fluid to or from reservoir 340.

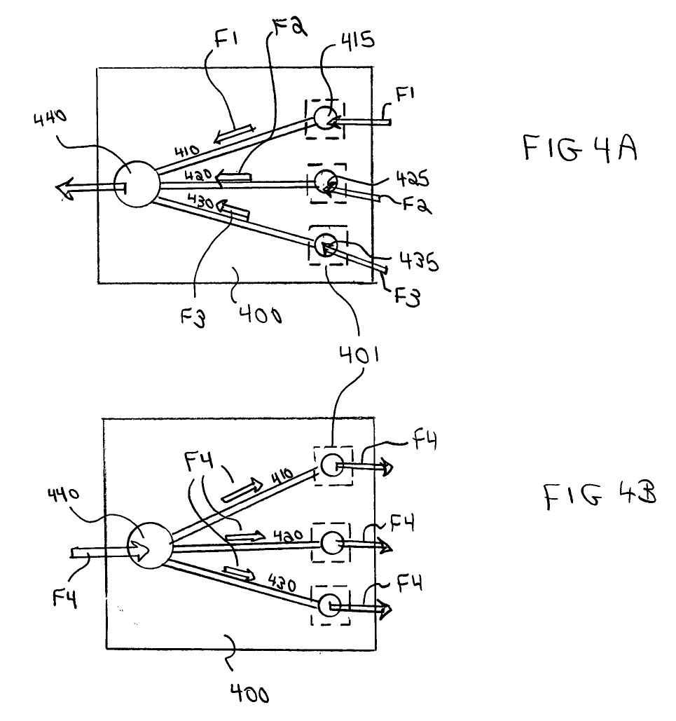

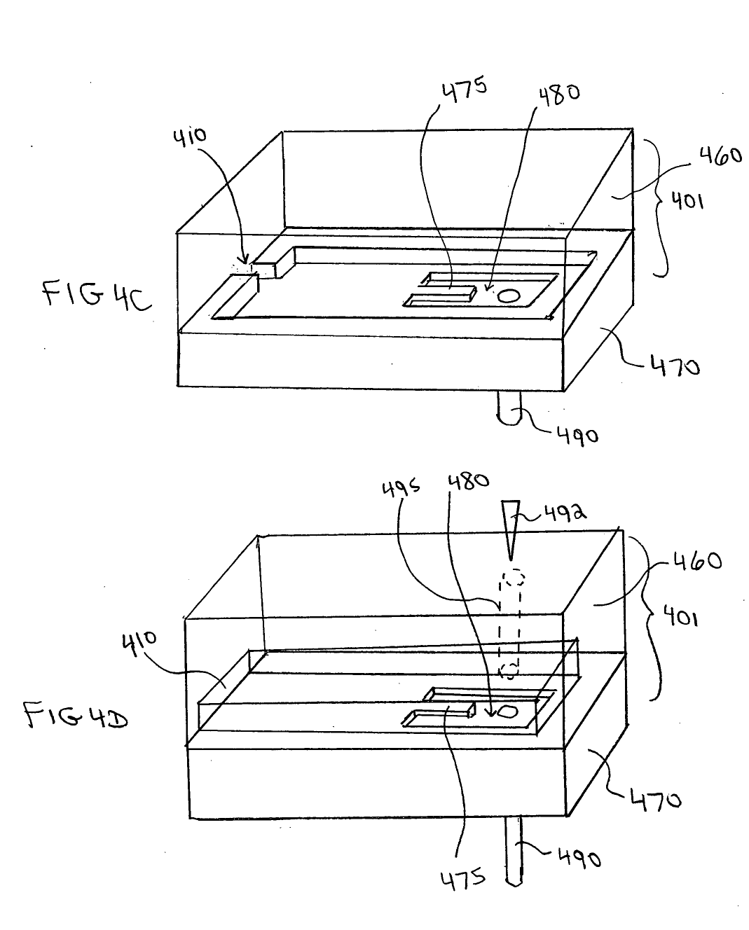

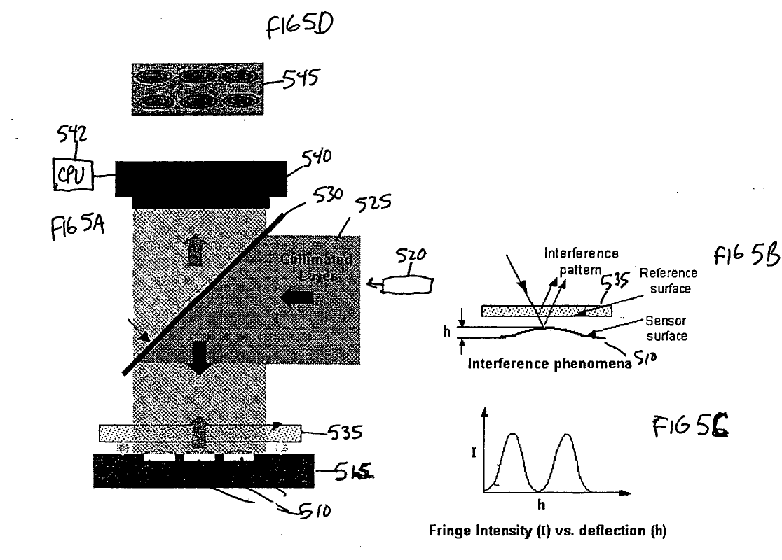

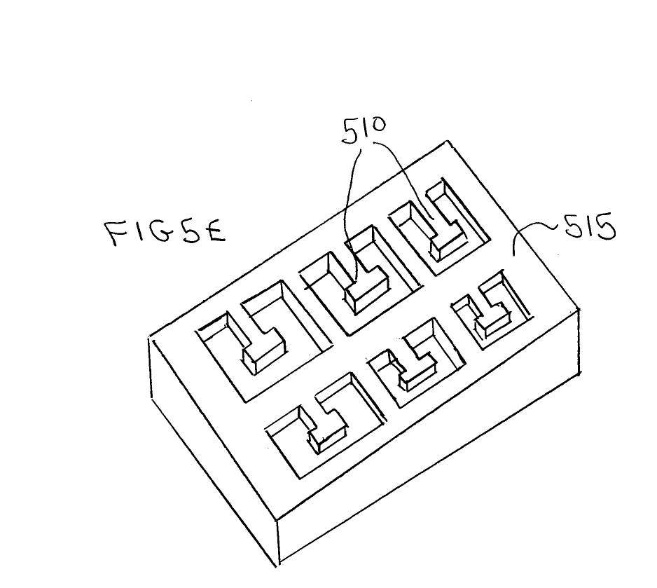

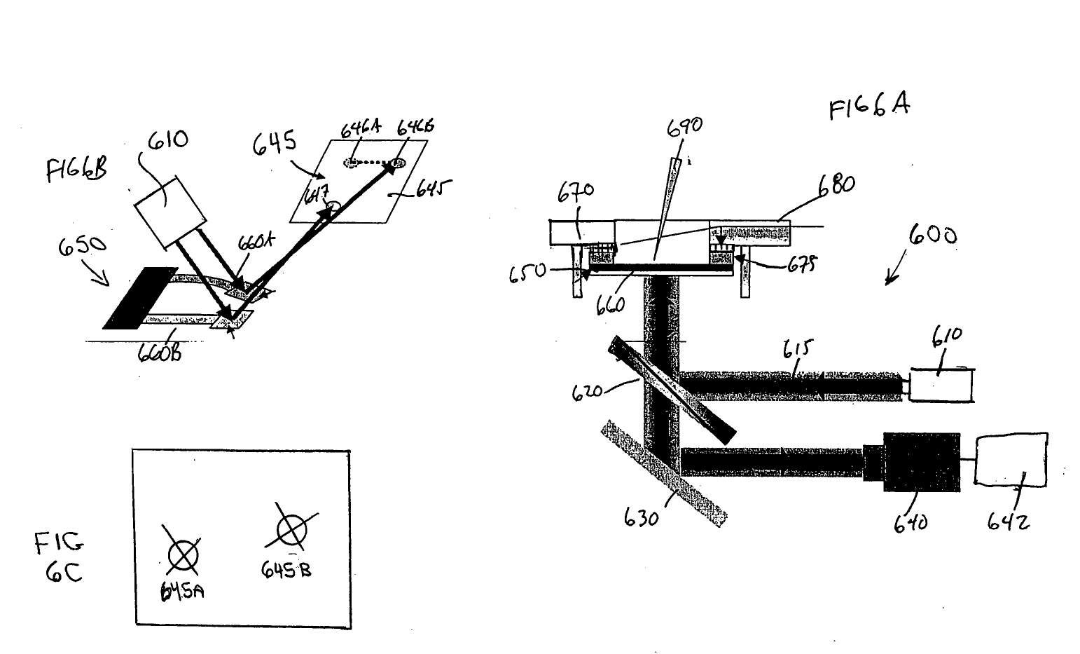

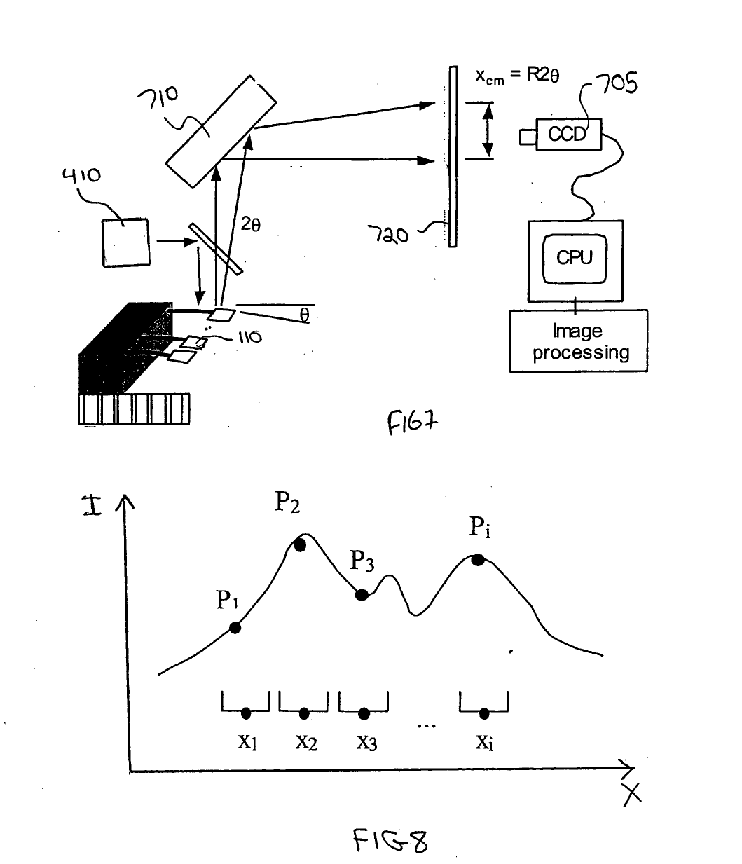

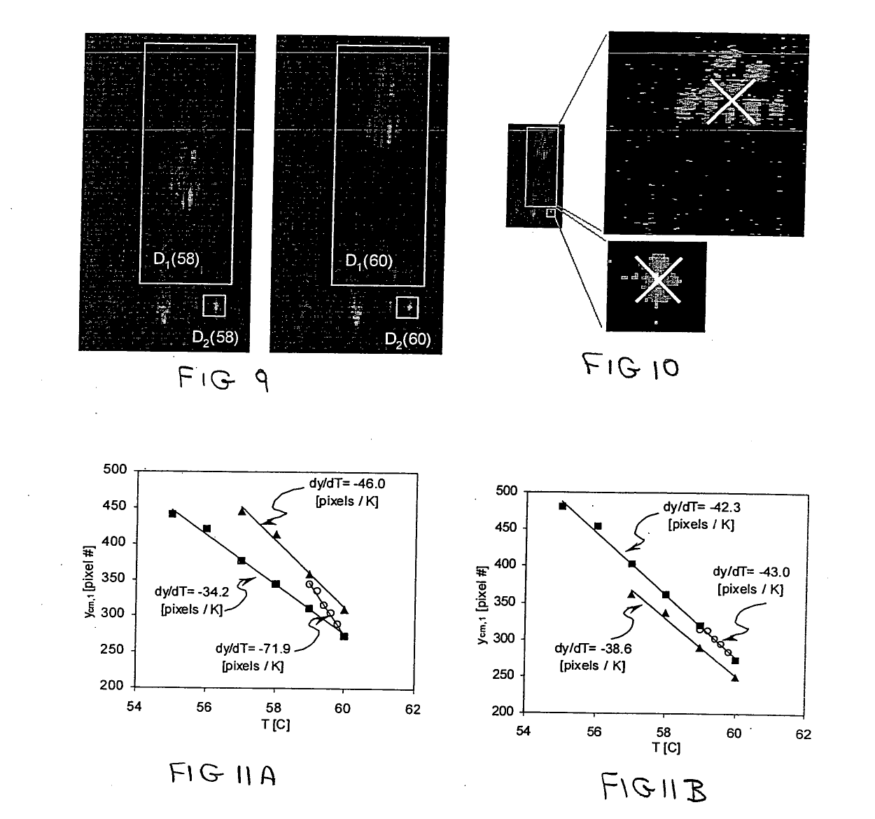

FIGS. 4A and 4B show various preferred embodiments of microfluidic systems with different sensors being functionalized to detect different target molecules (FIG. 4A) and with different sensors concurrently being exposed to the same fluid or gas for detection of different target molecules therein (FIG. 4B). FIGS. 4C and 4D are enlarged perspective views of first and second embodiments of a microfluidic cell 401 for use in the system of FIGS. 4A/4B.

Specifically, a target molecule may be detected using an assembly 400 including a glass portion 460 joined to a silicon portion 470. FIG. 4A shows a top view of fluid flow for functionalizing multiple sensors (in the example shown here, three) independently. Different functionalization fluids F1, F2 and F3 may be provided to three sensors (i.e.: a sensor in each of three cells 401 through holes 415, 425, and 435). The functionalization fluids F1, F2 and F3 flow through channels 410, 420, and 430, and then out of assembly 400 through a common hole 440. It is to be understood that the present invention comprises both embodiments with silicon portion 470 on the bottom of assembly 400 and glass portion 460 on the top of assembly 400, and vice versa.

FIG. 4B shows a top view of fluid flow for providing a sample material or for rinsing. Sample fluid F4 is provided into assembly 400 through hole 440, then to individual sensors in individual fluid cells 401 via channels 410, 420, and 430. Sample fluid F4 leaves assembly 400 via holes 415, 425, and 435.

FIGS. 4C and 4D show alternate preferred embodiments of microfluidic fluid cells 401. In FIG. 4C, cantilevers 475, reservoirs 480, channels such as channels 410, 420, and 430, and through holes such as holes 415, 425, and 435 are formed in silicon portion 470 of assembly 400. Fluid may be provided into the cell 401 via tubing 490.

In FIG. 4D, cantilevers 475 and reservoirs 480 are formed in silicon portion 470 of assembly 400. Channels such as channels 410, 420, and 430, and through holes such as holes 415, 425, and 435 are formed in glass portion 460 of assembly 400. Fluid may be provided into the cell 401 via channel 410 in glass portion 460. Alternatively, fluid may be provided into cell 401 of assembly 400 via a micropipette 492, using through holes 495 (shown in dotted lines) through glass portion 460. To introduce fluid using micropipettes, a robotic arm holding a micro-pipette array may be used, where the micro-pipette array may be commercially available. Each micro-pipette may contain a different probe molecule for functionalization, or at least some may contain the same probe molecule. The robot arm may lower, dropping a volume of solution into each hole. The probe molecules in the solution may reach the cantilevers by diffusion.

In other implementations, different portions of assembly 400 may be at least partially formed in glass portion 460, while others are at least partially formed in silicon portion 470. Alternate materials may be used for glass portion 460 and/or silicon portion 470.

It is to be understood that gasses may be substituted for fluids in all embodiments of the present invention. As such, the present invention may be used to detect target molecules in either fluids or gasses, all keeping within the scope of the present invention.

Although fabrication of microcantilevers has been described, other MSSS may be formed for detecting target molecules or biological or chemical reactions. Examples of MSSS and fabrication techniques are known; for example, see Microsensors MEMS and Smart Devices, Gardner et al., John Wiley & Sons, Ltd. (2001).

Referring to FIGS. 5A-5D, multiplexed detection of changes in a sensor array may be used to detect different molecules in a sample fluid at the same time, providing a significant efficiency improvement over existing systems. (Specifically, different sensors in the array are be functionalized to detect different target molecules). Additionally, multiplexed detection of changes in a sensor array may allow for detection of the same material in a number of different fluid or gas samples. In addition, a plurality of sensors may all be functionalized to detect the same target molecule. Multiplexed detection of such sensors may then be used to reduce the potential for detecting false positives. In other words, the presence of the target molecule may be confirmed by observing the effect on a plurality of sensors. When such a plurality of sensors are all functionalized to detect the same target molecule, these sensors may be disposed in either separate fluid reservoirs, or they may be disposed within the same fluid reservoir.

An optical interferometry system 500 may be used to detect changes in an array of sensors such as a membrane sensor array 510 on a chip 515. A laser 520 produces light, which is modified to produce a collimated beam 525 sufficient to illuminate sensor array 510. Beam 525 is incident on a beam splitter 530. A portion of beam 525 is directed toward sensor array 510. The portion is incident on a reference surface 535, which reflects part of the light and transmits part of the light. Some of the transmitted portion is reflected by the sensors of sensor array 510.

The sensors of sensor array 510 may be MSSS other than membranes. The sensors move in response to biological reactions. For example, cantilevers bend due to changes in surface stress. Therefore, detection methods that reflect the physical differences exhibited by the cantilever due to the change in surface stress (e.g., detection methods that detect the change in angle and/or deflection of the cantilever) may be used.

Similarly, the curvature of a membrane surface changes due to a change in surface stress caused by a biological reaction, as does the physical position of points on the membrane surface. Other MSSS may exhibit different physical movement in response to a biological reaction, which can be detected using optical methods.

For interferometry detection, light reflecting off different sensors with different positions (due, e.g., to a biological reaction of a target molecule with a probe molecule on a surface of the sensor) travels different path lengths. Referring to FIG. 5B, the surface of membrane 510 is deflected upward. The amount of deflection may change as target biological molecules react with probe molecules on membrane 510. As the position of the surface of membrane 510 changes, the reflected light interferes differently with the portion of light reflected from the reference surface, which travels a fixed distance. The resulting interference patterns may be detected to determine the presence and/or concentration of the target molecule.

FIG. 5C shows the relationship between fringe intensity and sensor deflection. The interference patterns may be detected using an optical detector such as a CCD camera 545. Different optical detection schemes may be used; for example, a CMOS array detector may be used. The output of CCD camera 545 may be analyzed using a CPU 542 for image processing.

FIG. 5D shows an array of interference patterns 545 that might result from an array of sensors 510 such as that shown in FIG. 5E. By tracking the changes in the intensity, the deflection of the sensor (e.g.: the microcantilever or membrane) may be determined.

Referring to FIGS. 6A-6C, multiplexed detection of changes in a sensor array may be accomplished using a ray-optics based experimental apparatus 600. A laser 610 provides a beam 615. Beam 615 is split by a beam splitter 620. A portion of the light is incident on an array of sensors 660 on a chip 650. Some of the incident light is reflected by the sensors, transmitted through beam splitter 620, reflected by a mirror 630, and detected using an optical detector 640. Optical detector 640 may be, e.g., a CCD camera or CMOS array detector. A CPU 642 may be used to receive and process data from optical detector 640. Apparatus 600 may also include a base 670, a heat sink/heat spreader 675, coolers 680, and micropipettes 690 for providing fluid to the apparatus.

FIG. 6B shows an expanded view of the detection scheme. Light is reflected from a first sensor (e.g., a functionalized cantilever) 660A and produces a spot on an image screen 645. When sensor 660A is not deflected (e.g., appreciably no target molecules have interacted with probe molecules), the spot is at position 646A. As time progresses, and target molecules react with probe molecules, the spot may move to position 646B.

A reference sensor (e.g., non-functionalized cantilever) 660B reflects light, which produces a spot 647 on screen 645. Spot 647 may remain in the same place, or may drift in position. Since the factors that may cause spot 647 to drift (e.g., temperature-induced deflection of the sensor) generally induce drift in spot 646 (the desired signal), the reference sensor can be used to for common mode rejection. That is, the difference in position between spot 646 and 647 may be used to determine the deflection of the functionalized sensor due to biological interaction between the probe and target molecules.

FIG. 6C illustrates the difference in position between two spots such as spot 646A and 646B of FIG. 6B, which may be determined by a centroid algorithm. This and other methods of determining the deflection of the functionalized sensor, as well as methods for increasing the signal to noise ratio, are described below.

As can be appreciated, the present invention provides systems and techniques that are especially useful for high-throughput molecular or biomolecular analysis. Such molecular or biomolecular analysis may include, but is not limited to, detecting biological interactions such as DNA hybridization, antigen-antibody binding, protein-protein interactions, and DNA-protein interactions, using the presently described sensors, which may include, but are not limited to, microcantilevers. Moreover, in accordance with the present invention, microfluidic and optical methods and apparatus are provided which enable multiplexed molecular or biomolecular analysis, and its corresponding benefits.

The presently described microfluidics may be used for selective functionalization of an array of cantilevers or other sensors, providing the capability to detect the presence of more than one type of molecule. Optical techniques described herein may be used to enable simultaneous detection of nanometer scale deflections of multiple cantilevers. Alternately, the present optical techniques can be used to detect physical change such as deflection of sensors other than cantilevers, such as membranes. Finally, common-mode rejection techniques are described for significant noise reduction.

(2) Integration of Microfluidics with Sensor Arrays:

An aspect of the present invention provides an N×M array of functionalized microcantilevers for DNA detection, protein detection or other detection, where at least one of N and M is greater than one (that is, multiple sensors are included). The N×M array may optionally be configured to sense the same target molecules or biomolecules, or may be configured to sense different target molecules. Specifically, the microcantilevers, functionalized with probe molecules, deflect when exposed to target molecules. This deflection is sensed using an optical technique described herein and can be used for DNA/protein detection. The device described herein may optionally include a silicon substrate such as a silicon wafer, and a glass substrate such as a glass wafer. Device features are fabricated in at least one of the substrates; for example, using MEMS fabrication techniques. This array can be used to detect multiple target molecules simultaneously.

(a) Fabrication of the Microarrays:

The device is fabricated such that the cantilevers are suspended in a reservoir as shown in FIG. 4. A silicon nitride layer is made on the silicon wafer, and the silicon nitride is patterned and etched so that of this layer, only the cantilever remains. The reservoir is then made by etching into the silicon. This reservoir not only allows for cantilever deflection, which enables the optical detection technique, but it also enables the fluid to fully surround the cantilever, enhancing functionalization. Microchannels are also fabricated to funnel fluid from the inlets and outlets to the cantilever reservoir. These microchannels can be fabricated by either etching the channels from the upper glass wafer (FIG. 4D) or by etching the channels from the silicon wafer on a plane above the cantilever prior to forming the cantilever (FIG. 4C) .

(b) Inlet/Outlet (I/O) Design:

As can be seen in FIGS. 4A to 4D, fluid can be introduced into microchannels 410, 420 and 430 at either end of assembly 400, depending on the flow path desired (i.e.: as shown in FIG. 4A or 4B). Microchannels 410, 420 and 430 preferably have small through-holes 415, 425 and 435 adjacent to sensors in associated fluid cells 401. At the opposite end of assembly 400, microchannels 410, 420 and 430 feed into one large hole 440, (See FIG. 4A). Fluid or gas may be introduced into through-holes 415, 425 and 435 to allow for individual functionalization of cantilevers 475.

After functionalization, the flow direction may be reversed by introducing flow into large hole 440. Introduction of fluid into large hole 440 results in flow in each of microchannels 410, 420 and 430 as also shown in FIG. 1B. This (opposite) direction of flow allows for simultaneous rinsing and detection for all of channels 410, 420 and 430. It is to be understood that the same fluid many be introduced into through-holes 415, 425 and 435; or, different fluids may be introduced into through-holes 415, 425 and 435, depending upon whether each of the sensors are to be functionalized to detect the same, or different, target molecules.

(c) Microfluidic System Design A:

The first addressing scheme is designed for an N×M array of detectors, where N and M may be on the order of about 1-10. In this scheme, the cantilevers are individually functionalized using microtubes (e.g. 490 in FIGS. 4C and 4D) attached to the individual through-holes at the base of the reservoirs (e.g. 480 in FIGS. 4C and 4D). By controlling the pressure on each of the tubes, the flow from the small through-holes through the channels can be controlled, allowing for functionalization without contamination between channels. Simultaneous rinsing and detection may also be performed by introduction of fluid through a tube (not shown) attached to the large hole 440 mentioned above.

(d) Microfluidic System Design B:

A second addressing scheme for the cantilevers would permit individual functionalization and simultaneous rinsing of many cantilevers—an N×M array, where N and M may be on the order of about 10-100. This array would allow for simultaneous detection of a large number of targets. This design is similar in fabrication and flow pattern to the previous design; however, the functionalization mechanism differs, allowing for a larger array of detectors. In this design, each of sensors (e.g. cantilevers) has a through-hole or passage below the cantilever well (e.g. a continuous passage from input 117 and exhaust 118 as shown in FIG. 1G) which may be used directly for functionalization without the use of microtubing. The probe molecules may be introduced to the cantilever sensor by means of a commercial micro-pipette array. A robotic arm may be used to hold the micro-pipette array, where each micro-pipette in the array may contain a different probe molecule for functionalization. Alternately, some or all of the micro-pipettes may contain the same probe molecules. The robot arm may lower, dropping a small volume of solution from the micro-pipettes on top of corresponding hole. In such an embodiment, the probe molecules in the solution may reach the cantilevers by diffusion. Like the previous design, channels may be connected, allowing for simultaneous rinsing and detection capabilities.

(3) Optical Readout of Cantilever Arrays

(a) Overview:

A split photodetector technique may be used to detect cantilever deflection. In this technique, one position sensitive detector (PSD) is used to monitor the angle of reflection of a laser beam off of a single cantilever. Other possible techniques for detecting small cantilever displacements include interferometry (Fabry-Perot and diffractive), piezoresistive strain gages, and the shift of mechanical resonance frequency of the cantilever as the mass of the cantilever changes due to adsorption of target molecules.

Using a charge coupled device (CCD) may provide an effective method for detecting light deflected from a sensor such as a cantilever. A CCD 705 may have an array of on the order of 10⁶ CCD pixels, and so provide sensitivity to small changes in intensity over a substantial area.

A schematic of this embodiment in a simple form is shown in FIG. 7. A low power He-Ne laser (λ=632.8 nm) is used to illuminate the sensors; here, five cantilevers 110 are used. For a cantilever 110 inclined at an angle θ from the horizontal, the reflection from the paddle 112 occurs at 2θ from the vertical, and is reflected once in mirror 710 before reaching an opaque imaging screen 720. The total path length from paddle 112 to screen 720 is R, and so the deflection of a spot on the screen is

s=2Rθ. (1)

In some implementations, screen 720 may be omitted and a single convex lens may be placed after the mirror to scale the images to fit directly onto the CCD array. This is a useful way to control the zoom ratio in order to make efficient use of the available CCD pixels. A custom temperature controller using thermistors and thermoelectric coolers may be used to control the temperature with precision and stability better than 10 mK.

The screen image may be captured by a CCD camera and transferred to a computer for analysis. The resulting data is generally an array of numbers representing the intensity of light at each CCD pixel. These numbers may be processed in different ways to obtain the deflections of multiple cantilevers 110.

(b) Center of Mass Algorithm:

Given a domain of pixels representing the light reflected from a cantilever, it is desired to relate deflections of the cantilever to changes in the pattern of light at the pixels. The center-of-mass (CM, really a center-of-intensity) approach is one method: The location of the CM of the light is taken to represent the coordinates of the reflection, and through simple geometry related to changes in angle at the cantilever tip itself. The optical power at each pixel in the domain of interest is represented as the 2D matrix Pᵢⱼ, where i and j cover the x and y directions, respectively. FIG. 8 shows a 1-D representation of intensity profile in a CCD array. Then the CM calculation is simply:

$$\begin{matrix}{x}_{cm} = \frac{{\sum }_{i , j} {P}_{ij} {x}_{ij}}{{\sum }_{i , j} {P}_{ij}} , & & {y}_{cm}\end{matrix} = \frac{{\sum }_{i , j} {P}_{ij} {y}_{ij}}{{\sum }_{i , j} {P}_{ij}} \qquad ( 2 )$$

For this formulation, noise present in any Pᵢⱼ is given equal weight, regardless of the particular pixel. This may be detrimental to the signal-to-noise (SNR) ratio; for example, if background pixels are included in the calculation that contain noise but do not include a contribution to the intensity due to cantilever reflection. Better results may be achieved by keeping the domains as small as possible, while encompassing substantially all of the cantilever reflection. A second method that may be used to boost SNR is to apply threshold criteria. For example, if Pᵢⱼ<10% of full scale, set Pᵢⱼ=0. This reduces the contributions of background noise at pixels that don't have much signal to begin with.

FIG. 9 shows the effect on the cantilevers of a change in temperature. Specifically, FIG. 9 shows representative CCD images of the bank of five cantilevers at 58° C. (left) and 60° C. (right). In both images there are bright, static spots at the bottom of the picture, including the one in the domains marked D₂. The domains D₁ contain the portion of the image due to the movement of the cantilevers. The displacement caused by this change of 2 K is clearly visible by eye. FIG. 10 shows the two domains of the 60° C. image that have been processed by the center of mass algorithm. The white “X” in each sub-image marks the calculated center-of-intensity within that domain.

An image processing algorithm was used to track the center of intensity within domain D₁ for three temperature sweeps in the range 55-60° C. (FIG. 11A). The sensitivity in terms of pixels ranges from 34 to 72 pixel/K, with the fit of each of the three lines separately being quite reasonable. There is also a small component in the x direction of less than 2 pixels/K (not shown). Making this assumption, the data of FIG. 11A were modified by fitting for the constant drift rate [pixels/minute] that minimizes the disagreement in slopes. FIG. 11B shows the results. For an assumed drift of +1.85 pixels/min, the revised temperature sensitivity becomes −41.3±5 pixels/K. The variation between the three slopes has been reduced by nearly an order of magnitude. Furthermore, this revised value corresponds to −0.68±0.09 μm/K at the paddle tip, which is within error of the other experimental value.

(c) Gain Factor Algorithm:

For very small deflections under low-drift conditions, the motion of the reflected spot on the CCD array may be very small. For example, a cantilever deflection of 30-50 nm might correspond to only a few pixels of change in x_{cm}, whereas the spot itself might be 50 pixels or more in diameter. In this situation, when ΔPᵢⱼ<<Pᵢⱼ, another algorithm may be employed that should improve the SNR.

For small deflections the response at any one pixel is assumed to be linear:

$$\frac{\partial {P}_{ij}}{\partial κ} = {const}_{ij} \qquad ( 3 )$$

where κ represents any parameter that is approximately linear with the cantilever deflection, such as curvature, tip deflection, tip angle, or surface stress. The constant constᵢⱼ can be different at every pixel. After calibration, even a single pixel can be used to calculate changes in deflection from changes in Pᵢⱼ. For a better SNR, multiple pixels may be used in the calculation, and more weight may be given to those that have high sensitivity and low noise. For a certain step change at the cantilever, Δκ, the signal can be defined as:

$$S = β {\sum }_{i , j} {g}_{ij} Δ {P}_{ij} \qquad ( 4 )$$

where gᵢⱼ is a gain factor to be applied to the change in intensity ΔPᵢⱼ at each pixel i,j and β is an arbitrary calibration constant. The gᵢⱼ are chosen to optimize SNR as discussed below.

Two common sources of noise are photon shot noise, Nᵢⱼ∝gᵢⱼPᵢⱼ^{1/2}, and constant background noise Nᵢⱼ=gᵢⱼ* constant. Examples of the latter may include fluctuations in ambient light, or the inherent CCD uncertainty of ±1/2 of one least significant bit (LSB). These can all be expressed as special cases of the more general form

Nᵢⱼ=A gᵢⱼPᵢⱼ^{γ}, (5)

where A is a known constant and the exponent γ is 1/2 for shot noise and 0 for constant background noise. Because the noise at each pixel is substantially uncorrelated with any other pixel, the total rms noise is found by adding the individual sources in quadrature:

$$N = {N}_{rms} = {( {A}^{2} {\sum }_{i , j} {g}_{ij}^{2} {P}_{ij}^{2 γ} )}^{1 / 2} . \qquad ( 6 )$$

Using the parameters from equations (4) and (6), the gain factors may be found by maximizing SNR with respect to each of the gᵢⱼ. Applying the extremum condition,

$$\frac{\partial SNR}{\partial {g}_{ij}} = 0 ,$$

for all i, j in the domain of interest, leads to the requirement

$${g}_{ij} = λ ( \frac{Δ {P}_{ij}}{{P}_{ij}^{2 γ}} ) \cdot ( \frac{{\sum }_{i , j} {g}_{ij}^{2} {P}_{ij}^{2 γ}}{{\sum }_{i , j} {g}_{ij} Δ {P}_{ij}} ) . \qquad ( 7 )$$

where λ is an arbitrary constant introduced because the SNR is independent of any multiplier that applies to all gᵢⱼ equally. Note that the second term in brackets is another constant value independent of the particular pixel i,j being considered (because the summations run over all pixels). Therefore we are free to set λ as the inverse of this quantity and write

$${g}_{ij} = ( \frac{Δ {P}_{ij}}{{P}_{ij}^{2 γ}} ) . \qquad ( 8 )$$

A further generalization is to deal with multiple uncorrelated noise sources, each with form of Equation (5). The rms noise then becomes

$${N}_{rms} = {\sum }_{k} {( {A}_{k}^{2} {\sum }_{i , j} {g}_{ij}^{2} {P}_{ij}^{2 γ ( k )} )}^{1 / 2} \qquad ( 9 )$$

where now the coefficients Aₖ and exponents γ_{κ} depend on the nature of the k^{th} type of noise source. The analogous form of equation (8) is then

$${g}_{ij} = ( \frac{Δ {P}_{ij}}{{\sum }_{k} {A}_{k}^{2} {P}_{ij}^{2 γ ( k )}} ) . \qquad ( 10 )$$

The final piece of the method is to obtain the calibration constant β. A known deflection Δκ_{cal} is applied, the resulting ΔP_{cal ij} recorded, and Equation (8) or (10) used to set the gᵢⱼ. After these gᵢⱼ have been fixed they remain constant for all subsequent measurements. For the calibration run, equating S of equation (4) to Δκ_{cal} gives

$$β = \frac{Δ {κ}_{cal}}{{\sum }_{i , j} {g}_{ij} Δ {P}_{cal ij}} . \qquad ( 11 )$$

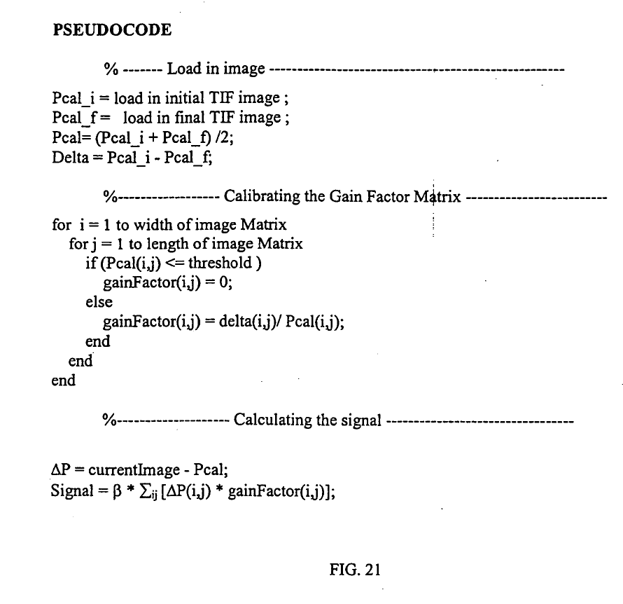

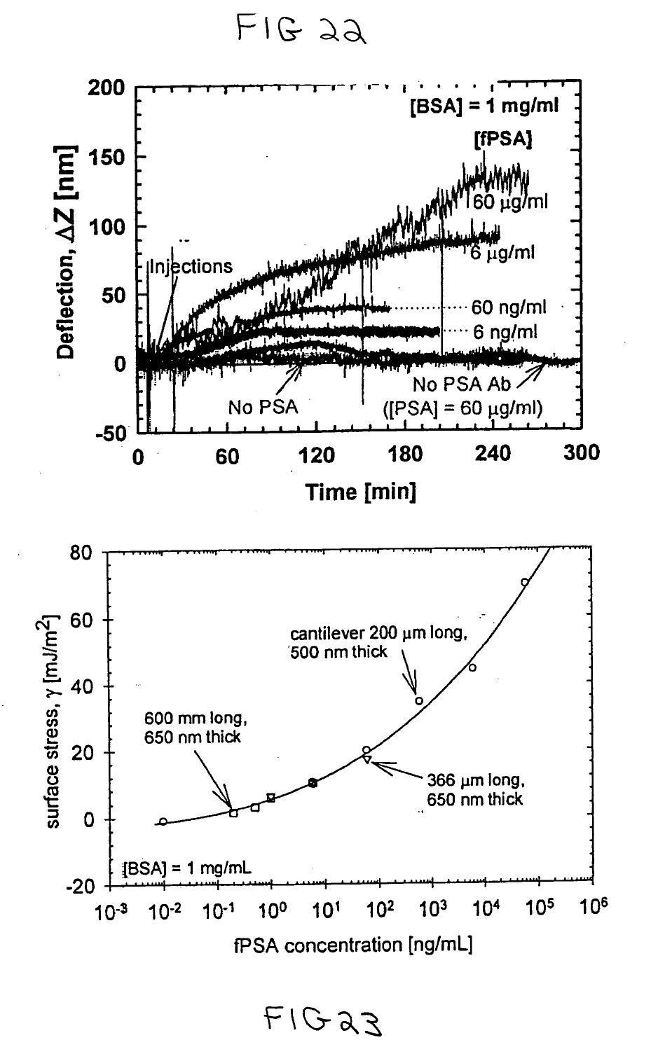

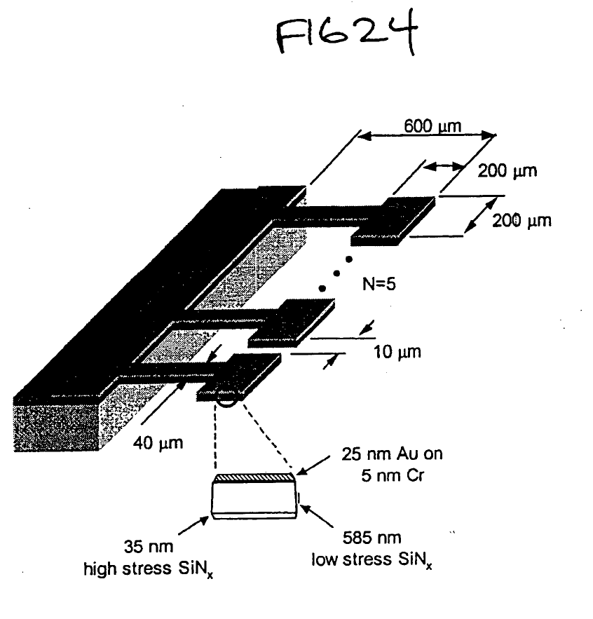

Using MATLAB, Equation (8) was applied to some recent data to validate the algorithm using γ−1/2. FIG. 20 provides a flow chart and FIG. 21 provides pseudocode of this calculation. One problem with this case is the possibility for division by zero at the background pixels where Pᵢⱼ is small. To avoid this, a threshold criterion may be applied to force gᵢⱼ to zero when Pᵢⱼ itself is sufficiently small.

FIGS. 12 to 16 are images illustrating the various stages in the process of calculating the gain factors. For this series, images were taken for 20 minutes at 2 minute intervals, and the calibration process was applied to the two endpoint images.

FIG. 12. Pcal_i=Initial image for ΔP calibration

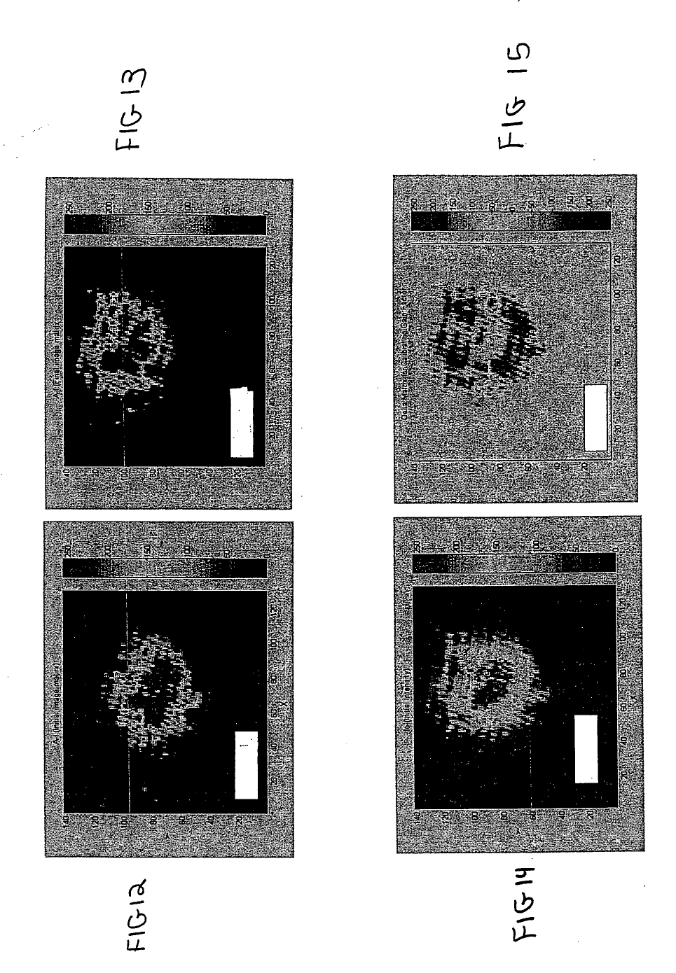

FIG. 13. Pcal_f=Final image for ΔP calibration

FIG. 14. Pcal=average of initial and final images

FIG. 15.

Delta=initial−final.

FIG. 16.

Gain factor matrix=Delta./Pcal.

In this example β was not calculated independently and so the resulting signal S is in arbitrary units.

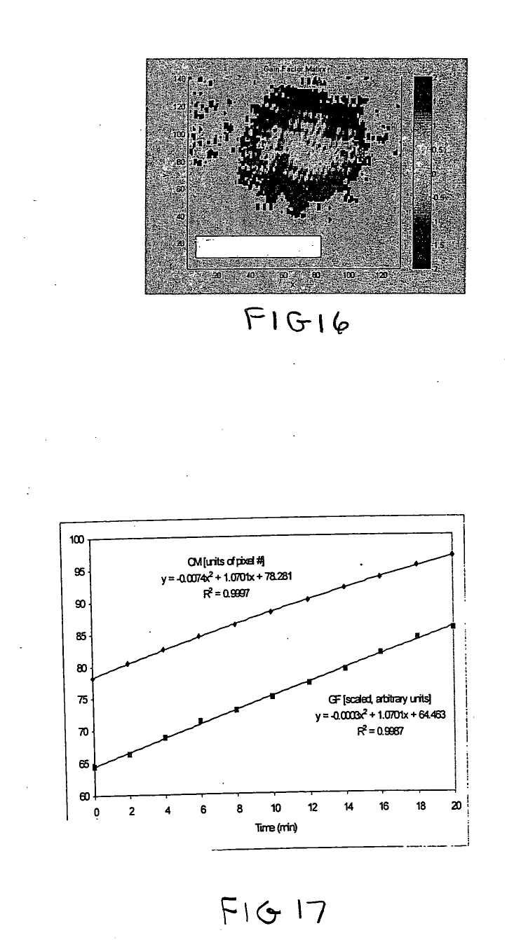

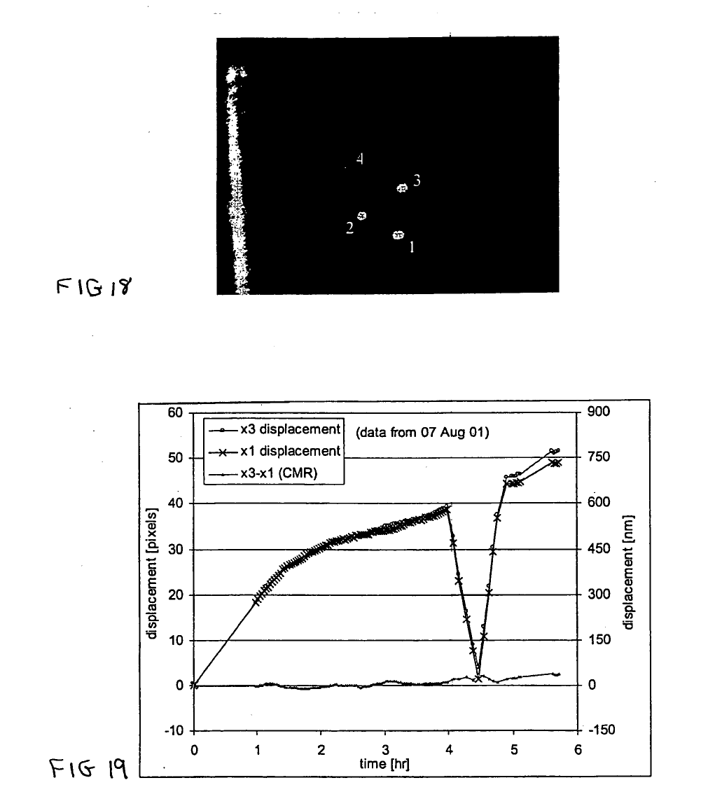

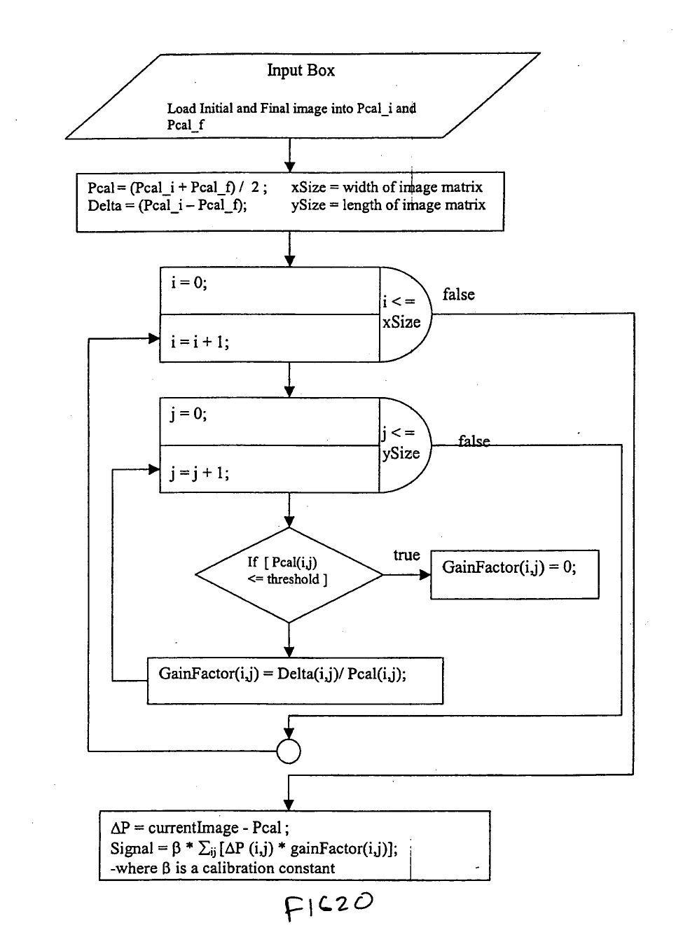

A comparison of the Center of Mass and Gain Factor algorithms is shown in FIG. 17. Assuming the drift over this timescale can be modeled as a parabolic variation with time, the CM method may be better than the GF method for this data, as indicated by the R² goodness of fit. This is not surprising considering that the motion of the spot is about 20 pixels, which is not negligibly small compared to the spot size of approximately 50 pixels.

(d) Slope Method:

In the case where the shape of the intensity profile does not vary substantially with time, it is not necessary to use a two point calibration with known Δκ_{cal} in order to calculate the calibration constant β. Instead, the relationship between the spot location and κ is:

X_{cm}=c·κ+X_{ref}.

where c may be found using, e.g., geometric optics or past calibrations. If the profile shape is taken from a single image and assumed constant, then the changes in Pᵢⱼ are theoretically known for any small change in x_{cm} (or equivalently κ) by using the gradient of the profile in the direction of motion. That is,

$$Δ {P}_{cal ij} = - c \cdot {[ \frac{\partial {P}_{cal}}{\partial x} ]}_{x = x ( ij )} Δ {κ}_{cal} . \qquad ( 12 )$$

Extending this to the gain factors and calibration constant:

$$\begin{matrix}{g}_{ij} = \frac{- {[ \frac{\partial {P}_{cal}}{\partial x} ]}_{x = x ( ij )}}{{P}_{ij}^{2 γ}} , & ( 13 ) \\ β = \frac{1}{c \cdot {\sum }_{i , j}^{} {{g}_{ij} [ \frac{\partial {P}_{cal}}{\partial x} ]}_{x = x ( ij )}} & ( 14 )\end{matrix}$$

This method has not yet been tested on any experimental data. Caution should be used in evaluating the gradients

$$\frac{\partial {P}_{cal}}{\partial x}$$

numerically, because differentiation may enhance noise.

(4) Common-Mode Rejection

The present invention also provides for multiplexed monitoring of multiple cantilevers (or other sensors) that may be used to obtain a high throughput detection device. Additionally, by providing systems and methods for multiplexed monitoring, the present invention optionally includes reference sensors that may be used for rejection of common mode drift, as follows.

Cantilever systems may exhibit non-thermal drifts at the cantilever tip of one micron or greater, far larger than the expected biological deflections (e.g., on the order of several tens of nanometers). Accordingly, when the drift affects nearby cantilevers in the same way, the present invention provides use of a non-functionalized reference cantilever as a moving baseline. Specifically, if an adjacent cantilever is functionalized, then the biological signal can be taken as the difference between the displacement of the functionalized cantilever and the adjacent drifting control cantilever. It is to be understood that, in various embodiments, multiple cantilivers may be functionalized and compared to a single non-functionalized control cantilever. In addition, the displacement of one or more functionalized cantilevers can be compared to the displacement of one or more non-functionalized cantilevers, all keeping within the scope of the present invention. The use of multiple functionalized and/or non-functionalized cantilevers can also be used to reduce the likelihood of false positive detection, as follows. The detected deflection of multiple functionalized cantilevers can be compared to one another. In addition, the deflection of multiple non-functionalized cantilevers can be compared to one another). Functionalization of adjacent cantilevers can be done using microfluidics described earlier, and deflection detection of adjacent cantilevers can be achieved using the optics described earlier. The following describes an example.

FIG. 18 shows the reflections from four cantilevers in air, the rightmost pair of which were used with the center-of-mass processing algorithm to validate common-mode rejection. Over the course of nearly six hours, the pair of spots drifted by about 50 CCD pixels, corresponding to approximately 750 nm of tip deflection for this setup (FIG. 19). Specifically, motion of two spots (x3 and x1) is shown, with the curve x3-x1 showing Common Mode Rejection. The temperature was 66.5° C. for all points except the sharp “V” during hour 4, which corresponds to a gradual sweep to 67.5° C. and back. The differential displacement between the two spots was constant within one pixel (15 nm tip deflection) for the first 4 hours and within 3 pixels (45 nm) after that. This is an improvement from the single-cantilever drift. To restate, for a four-hour biological experiment without a reference cantilever but exhibiting drift of this magnitude, the uncertainty in tip deflection would be about ±600 nm, whereas using a reference for common mode rejection reduces this uncertainty by a factor of 40 to ±15 nm.

Thus, the present invention provides a device that allows for the parallel detection of multiple biomolecules through the binding-induced deflection of microcantilevers or other MSSS.

Below, studies of a bank of five cantilevers that are deflected due to thermal bimorph effects are discussed. Thermal bimorph effects may be used to model the changes in surface stress that are caused by molecular binding. The following illustrates an example of a system for detecting molecule using sensors such as cantilevers. Other systems and techniques are possible, all keeping within the scope of the present invention.

To demonstrate multiplexed detection, a bank of five cantilevers (each 600 μm long, 645 nm thick) is actuated thermally over a range of 3 μm of tip deflection. The cantilevers are illuminated with a laser and the resulting pattern of reflected spots is detected. For example, CCD-based detection may be used to detect the deflections of multiple cantilevers. A stress-balancing fabrication technique may also be used to reduce the initial deflection (e.g., the zero-point deflection) significantly; e.g., by an nearly order of magnitude.

(5) Operation of the Present Invention:

(a) Introduction:

In accordance with the present invention, molecules may be detected using sensors such as microcantilevers. When target molecules bind specifically to probe molecules attached to a surface of a microcantilever, there is a change in surface stress. The change in surface stress causes the cantilever to bend (FIG. 2). The probe molecules might be (but are not limited to) e.g., a short single-stranded DNA (ssDNA) sequence or an antibody, and may be attached to a substrate such as a gold substrate through, e.g., an intermediate thiol or cysteamine group. Once the probes have been attached, the cantilever may detect the targets (e.g., complimentary ssDNA, antigen). When present, target molecules may bind with the probe molecules, and the resulting change in surface stress is generally a function of analyte concentration. This phenomenon may used to detect DNA hybridization and protein-protein binding, for example. FIG. 22 shows some binding curves for various concentrations of the important cancer marker known as free prostate specific antigen (fPSA). fPSA is correlated with an overactive prostate and enhanced risk of prostate cancer when present in human serum at levels around 4-10 ng/mL.

The surface stress on the cantilever generally changes with the free energy changes occurring with biological reactions. Therefore, detecting changes in a cantilever or other sensor may be used to study a wide variety of biomolecular interactions, including DNA hybridization, DNA-protein, protein-ligand, etc. In addition, this method does not require fluorescent or radioactive labels, making it applicable to a broad range of biomolecules without affecting their native structure.

Multiplexed detection of changes in multiple sensors can provide benefits not found in previous efforts, which generally included a single cantilever, probed for a single molecule per trial, and lasted for times on the order of an hour. In contrast, a multiplexed system such as described here may allow for detection of many different biomolecular interactions at the same time. Since there are many thousands of biomolecular interactions possible in a living cell, the benefits of multiplexed detection may be significant. In order to perform multiplexed detection, the present systems and techniques may include common mode rejection, microfabrication of sensor arrays, and optical techniques to detect changes in multiple sensors. The present systems and techniques may allow detection of deflections of multiple cantilevers with nanometer scale resolution.

(b) Nomenclature:

Dᵢ domain on CCD image where spot of i^{th} cantilever lies

Eⱼ modulus of elasticity of j^{th} layer of cantilever [Pa]

N total number of cantilevers under consideration

R path length from cantilever to imaging screen [m]

T temperature [K]

X distance along cantilever from base [m]

Z cantilever deflection [m]

s displacement of a spot on imaging screen [m]

tⱼ thickness of j^{th} layer of cantilever [m]

xᵢ coordinate on CCD image of i^{th} cantilever [pixel #]

yᵢ coordinate on CCD image of i^{th} cantilever [pixel #]

α thermal expansion modulus of j^{th} cantilever layer [K⁻¹]

γ surface stress [J/m²]

κ curvature [m⁻¹]

θ angle of deflection of cantilever [rad]

σ residual stress in a thin film [N/m²]

υ Poisson ratio

(c) Mechanics of Stress-Induced Deflection:

The following analysis is applied to an exemplary bank of five cantilevers. For a homogeneous, prismatic beam, the relationship between surface stress and deflection is generally known as Stoney's Formula. For a slender beam with clamped-free end conditions,

$$γ = \frac{{E}_{1} κ {t}_{1}^{2}}{6 ( 1 - {v}_{1} )} \qquad ( 15 )$$

where γ is the surface stress, E is the modulus of elasticity, κ is the curvature, t is the thickness of the beam, υ is Poisson's Ratio (˜0.25 for silicon nitride, SiNₓ), and the subscript 1 denotes the thick, low stress SiNx layer of the beam. The (1−υ) factor in the denominator appears for a slender beam with clamped-free end conditions that is allowed to bend in two directions. Further assumptions are isotropic material properties and an aspect ratio (length/width) greater than about 5. This expression is also valid if there are other thin layers present as long as they do not significantly affect the composite beam's stiffness in bending. In our work, the surface stress of Eq. (15) has three important causes. The first cause is the biological reactions at the surface,

γ=γ_{bio}. (16)

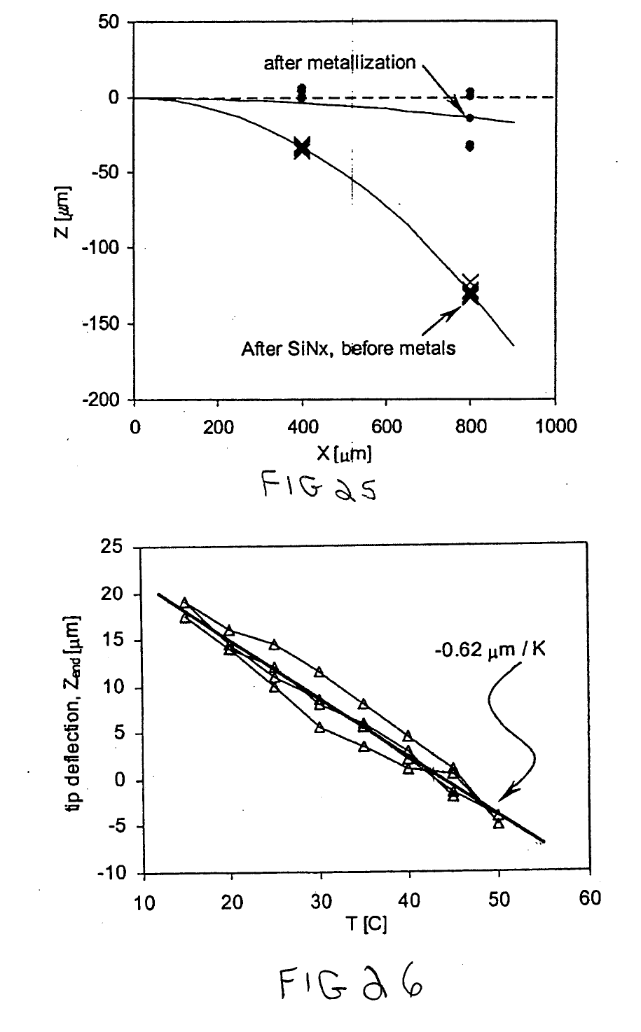

Because the reactions are limited to the surface, the changes in surface stress are generally independent of the geometry of the cantilever itself. FIG. 23 demonstrates this invariance for fPSA for three different cantilevers of various thickness and length. Note that in order to detect fPSA in the critical regime of 1-10 ng/mL, γ must be measured with an accuracy of ˜1 mJ/m².

Second is the thermal bimorph effect caused by the different moduli of thermal expansion α of the gold and nitride layers (14×10⁻⁶ K⁻¹ compared to 1×10⁻⁶ K⁻¹). The resulting temperature-dependent surface stress may be written as

γ=(α₂−α₁)ΔT E₂t₂. (17)

where ΔT is the temperature difference from some reference state, E₂ is the elastic modulus of the gold layer, and t₂ its thickness. This expression is valid in the limit t₁/t₂>>1 and (E₂/E₁)<<(t₁/t₂)². For our system of gold on nitride, t₁/t₂≈25 and E₂/E₁≈1, so this limit is appropriate. Although this temperature sensitivity may obscure signal due to biological reactions and therefore may be undesirable in practice, it can provide a benefit for prototype testing, since it allows us to model γ_{bio} in a much faster and more controllable fashion. For example, to achieve the biologically critical changes in surface stress of Δγ=1 mJ/m² with 25 nm gold (E₂=80 GPa) on SiNx (E₁=110 GPa [symbol] ), the temperature difference should be ΔT=39 mK, which is well within reach of modern temperature controllers.

A third source of surface stress is inherent in the deposition of thin films. For example, in the LPCVD furnace used in the UC Berkeley Microlab, the stress a of a SiNx film can be varied from ≈60−1000 MPa depending on the recipe and stochiometry. (Note the distinction between the familiar bulk stress σ [N/m²] and surface stress γ[N/m].) Again in the thin film limits of Eq. (17), this results in an effective surface stress of

γ=σ₂t₂, (18)

where σ₂ is the stress of the thin secondary film. Notice that Eq. (17) can be considered to be a special case of Eq. (18), where σ₂=(α₂−α₁) ΔT E₂. This forms the basis of the stress balancing technique used to counteract the tendency of the gold layer to pull the cantilever up. By judiciously choosing the thickness of the gold and high-σ SiNx films, the released composite cantilever can theoretically be made perfectly flat.

In general, the cantilevers will have some net curvature. From elementary small-deflection beam theory, for a beam with constant curvature the deflection Z(X) is simply parabolic:

$$\begin{matrix}Z = \frac{1}{2} κ {X}^{2} & ( 19 ) \\ θ = \frac{ⅆ Z}{ⅆ X} = κ X & ( 20 )\end{matrix}$$

where X is the distance along the cantilever measured from its base where Z and θ are zero, and Z is measured upwards from the perfectly flat condition. Thus a change in κ can be determined from measurements of cantilever deflection, and Eq. (15) used to calculate the corresponding change of surface stress.

(d) MEMS Design:

FIG. 24 is a schematic of the bank of cantilevers used in this study. There are five cantilevers in the group, each including a 200 μm square paddle located at the end of a 400 μm long ×40 μm wide beam. The beam and paddle both include a 35 nm thick high stress SiNx layer (σ₂≈1000 MPa) underneath a low stress nitride layer of thickness 585 nm (σ₁≈280 MPa). On top of both regions is 25 nm gold, with a 5 nm chrome adhesion layer in between. The paddle is generally located at the end of a long beam in order to achieve a significant change in angle for a change in surface stress to be measured. The paddle reflects a large portion of incident light, so that the intensity of the received signal is sufficient.

The details of a method of fabrication are shown in FIGS. 3A to 3F for a single cantilever, where the paddle has been omitted for clarity. In this example, all devices were fabricated at the UC Berkeley Microlab. To form a cantilever array, high and low stress nitride layers are grown in sequence in an LPCVD furnace on a single polished silicon <100> wafer. Large openings are patterned in the backside to act as a mask for a subsequent etch, and then the cantilevers are patterned on the frontside. Next is a wet KOH or TMAH etch through the wafer is performed to release the cantilevers. Because of the stressy nitride underlayer, at this stage the released cantilevers are bent downwards by many tens of microns. The last step is the evaporation of chrome and gold, using a physical mask for coarse patterning. In principle the metals could be patterned far more precisely by evaporating and patterning prior to the cantilever release; however there is some concern that the delicate metal films may not withstand the extended wet etch.

One feature of the current systems and techniques is the use of a thin underlayer of high stress SiNx, which allows for “stress balancing.” Without this layer, the finished cantilevers would generally bend upwards due to the thermal and intrinsic stresses of the chrome and gold layers. Measuring the resulting curvature and knowing the value of σ2 for the high-stress SiNx, Eq's (15) and (18) may be used to calculate the thickness of stressy nitride that may be used to balance the stress of the metal layers. However, non-uniform fabrication may limit the effectiveness of this technique.

(6) Experimental Apparatus and Detection Scheme:

(a) Possible Detection Schemes:

A split photodetector technique may be used to detect cantilever deflection. In this technique, one position sensitive detector (PSD) is used to monitor the angle of reflection of a laser beam off of a single cantilever. Other possible techniques for detecting small cantilever displacements include interferometry (Fabry-Perot and diffractive), piezoresistive strain gages, and the shift of mechanical resonance frequency of the cantilever as the mass of the cantilever changes due to adsorption of target molecules.

Using a charge coupled device (CCD) may provide an effective method for detecting light deflected from a sensor such as a cantilever. A CCD may have an array of on the order of 10⁶ CCD pixels, and so provide sensitivity to small changes in intensity over a substantial area.

(b) Apparatus for This Study:

A schematic of the experimental arrangement in a simple form is shown in FIG. 7. A low power He-Ne laser (λ=632.8 nm) is used to illuminate the five cantilevers under test. For a cantilever inclined at an angle θ from the horizontal, the reflection from the paddle occurs at 2θ from the vertical, and is reflected once in a mirror before reaching an opaque imaging screen. The total path length from paddle to screen is R, and so the deflection of a spot on the screen is

s=2Rθ. (21)

Alternately, the screen may be omitted and an optical system such as a single convex lens may be placed after the mirror to scale the images to fit directly onto a CCD array. This may be a useful way to control the zoom ratio in order to make efficient use of the available CCD pixels. A temperature controller using thermistors and thermoelectric coolers is used to control the temperature with precision and stability better than 10 mK. As stated above, changing temperature is used to model changes that would occur due to biological reactions of probe molecules with target molecules.

The screen image is captured by a CCD camera (e.g., Cohu model 2122, 8 bit, 813×1008 pixels) and transferred to a computer for analysis (e.g., Hammamatsu/Argus Image Processor). The resulting image consists of a 1008×813 array of numbers representing the intensity of light at each CCD pixel. A short MATLAB program was written to calculate the locations of the reflected spots using a simple center-of-mass type of calculation. The algorithm is as follows:

1. Set system to temperature T

2. Grab one CCD image

3. High pass filter in amplitude by setting all pixels with intensity below 25% of full scale to zero.

4. For each cantilever i:

a. determine the smallest domain Dᵢ(T) within which the light from that cantilever must lie.

b. calculate the center of intensity like a center of mass:

$${x}_{c m , i} ( T ) = \frac{{\sum }_{j ∈ {D}_{i}}^{} {I}_{j} ( T ) {x}_{j}}{{\sum }_{j ∈ {D}_{i}}^{} {I}_{j} ( T )} , {y}_{c m , i} ( T ) = \frac{{\sum }_{j ∈ {D}_{i}}^{} {I}_{j} ( T ) {y}_{j}}{{\sum }_{j ∈ {D}_{i}}^{} {I}_{j} ( T )} \qquad ( 22 )$$

For the i^{th} cantilever, the summation is performed over all pixels in the domain Dᵢ. Here Iⱼ(T) is the intensity of the jth pixel and x_{cm,i}(T), y_{cm,i}(T) are the coordinates of cantilever i at temperature T in terms of pixel numbers.

c. repeat for all N cantilevers of interest.

5. Repeat for next temperature of interest.

The output of this algorithm is the temperature-dependent coordinates of each spot. Knowing the total path length R from cantilever to imaging screen, and having calibrated the CCD pixels in terms of length at the imaging screen, changes in curvature or surface stress at the cantilever may easily be calculated. There may be a tradeoff between high zoom for maximum resolution, and low zoom to image more cantilevers at once.

(7) Experimental Results

(a) Stress Balancing:

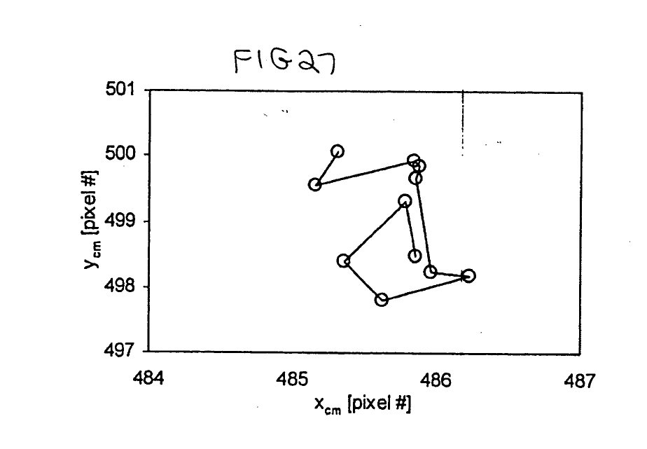

In order to validate the stress balancing technique, several test cantilevers were fabricated of length 400 and 800 μm and various thickness of the high stress SiNx underlayer. Representative results are shown in FIG. 25 for t₂=24 nm. After release and before metallization, the cantilevers are greatly bent down (X's). The line is a fit of Eq. 19 with a radius of curvature κ⁻¹=2.45 mm, showing that the model of parabolic deflection is an excellent one. After the metals are evaporated, the cantilevers are pulled back up close to the neutral position (solid circles) and the radius of curvature is improved by almost an order of magnitude to 23.3 mm. In this case the cantilevers remained bent slightly downwards, suggesting that the stressy nitride should be slightly thinner. The scatter in these data represent non-uniformity across a wafer and from two trials of the evaporation process.

(b) Image Processing:

FIG. 9 shows representative CCD images of the bank of five cantilevers at 58° C. (left) and 60° C. (right). In both images there are bright, static spots at the bottom of the picture, including the one in the domains marked D₂. The domains D₁ contain the portion of the image due to the movement of the cantilevers. The displacement caused by this change of 2 K is clearly visible by eye. It is thought that the distinct streaks within D₁ are due to the five different cantilevers; however, this has not been verified and so all subsequent calculations lump the entire bank of cantilevers as one moving spot. In practice, separate signals would be obtained for each cantilever.

FIG. 10 shows the two domains of the 60° C. image that have been processed by the center of mass algorithm. The white “X” in each sub-image marks the calculated center-of-intensity within that domain.

(c) Temperature-Induced Deflection:

As an independent check on the temperature sensitivity, an optical microscope was used to focus on the tip of one cantilever while a temperature controller made several sweeps between 15° C. and 50° C. in steps of 5 K. At each temperature the cantilever deflection was determined by noting how much the microscope stage had to be raised or lowered to bring the cantilever back into focus (FIG. 26). The best fit line gives a sensitivity dZ_{end}/dT=−0.62±0.05 μm/K.

For comparison, Eq's (15), (18), and (20) can also be used to predict the sensitivity. Using dimensions and property values already given, the calculated sensitivity is −0.50 μm/K. The uncertainty in this number is difficult to estimate because the material properties of thin films are not well characterized and the thickness sensor for metal evaporation was past its design lifetime. An error of 50% would not be surprising.

Note that a more universal parameter would be to express the sensitivity in terms of surface stress because it is independent of cantilever geometry, but we present our data in terms of tip deflection because it is more intuitive. For comparison, the important threshold value of Δγ=1 mJ/m² should correspond to a tip deflection of ΔZ_{end}=19 nm in these cantilevers, and a temperature change of about ΔT=40 mK.

The image processing algorithm was used to track the center of intensity within domain D₁ for three temperature sweeps in the range 55-60° C. (FIG. 11A). The sensitivity in terms of pixels ranges from 34 to 72 pixel/K, with the fit of each of the three lines separately being quite reasonable. There is also a small component in the x direction of less than 2 pixels/K (not shown). Applying a rough calibration to convert pixels to distance at the screen (260±30 μm/pixel), and using Eq's (19), (20), and (21) with R=2.88 m, the sensitivity at the cantilever tip is found to range from −0.56 to −1.19 μm/K. Although in the same neighborhood as the theoretical and other experimental value, the large variations far exceed the experimental uncertainty estimated at 10%. The data points shown span about 67 minutes and unexplained monotonic non-thermal drifts over this timescale have previously been observed by our group. Therefore one likely cause of some of the variation in FIG. 11A is a slow cantilever drift in the y direction.

Making this assumption, the data of FIG. 11A were modified by fitting for the constant drift rate [pixels/minute] that minimizes the disagreement in slopes. FIG. 11B shows the results. For an assumed drift of +1.85 pixels/min, the revised temperature sensitivity becomes −41.3±5 pixels/K. The variation between the three slopes has been reduced by nearly an order of magnitude. Furthermore, this revised value corresponds to −0.68±0.09 μm/K at the paddle tip, which is within error of the other experimental value.

As a check to confirm that the drift occurs at the cantilevers and not some other aspect of the experimental apparatus, the center of intensity of the putatively fixed spot in D₂ was also tracked. As shown in FIG. 27, any motion was limited to about one pixel in any direction. This includes mechanical drift of the test chip and optics, errors in the image processing hardware, and uncertainty introduced by image noise into the processing algorithm, thereby confirming that the drift seen in FIG. 11 is indeed due to the cantilevers themselves.

(d) Conclusions:

The present invention provides a multiplexed device that may be used to detect many biomolecules simultaneously. Thermal actuation has been used to validate a new CCD-based optical detection technique capable of tracking the deflections of many cantilevers simultaneously. The temperature sensitivity as calculated by the image processing algorithm is dZ_{end}/dT=−0.68 μm/K, which is within error of both another experimental value (−0.62 μm/K) and a theoretical prediction (−0.50 μm/K).

A number of embodiments have been described. Nevertheless, it will be understood that various modifications may be made without departing from the spirit and scope of the invention. For example, different types of sensors may be used, as well as different detection methods. Accordingly, other embodiments are within the scope of the following claims.

What is claimed is:

1. A system for detecting target molecules, comprising:
a plurality of sensors, each of the sensors including one or more associated probe molecules for interacting with the associated target molecules, each of the sensors exhibiting a physical change upon interacting with the associated target molecules; and
an optical detector, the optical detector positioned to receive a reflected beam of light from each of the plurality of sensors, wherein a property of the reflected beam of light for each of the plurality of sensors changes when the associated probe molecule interacts with the associated target molecules.

2. The system of claim 1, wherein each of the plurality of sensors include the same associated probe molecules for interaction with the same target molecules.

3. The system of claim 1, wherein each of the plurality of sensors include different associated probe molecules for interaction with different target molecules.

4. The system of claim 1, wherein the target molecules comprises at least one of the group consisting of:
a short single-stranded DNA sequence, an antigen, and a protein.

5. The system of claim 1, wherein the optical detector includes an optical sensor array.

6. The system of claim 5, wherein the optical sensor array comprises a CCD array.

7. The system of claim 5, wherein the optical sensor array comprises a CMOD array detector.

8. The system of claim 1, wherein the plurality of sensors comprises a plurality of micromechanical surface stress sensors (MSSS).

9. The system of claim 8, wherein the plurality of MSSS includes a microcantilever.

10. The system of claim 9, wherein the microcantilever comprises a reflective paddle portion.

11. The system of claim 10, wherein the reflective paddle portion comprises at least one strengthening ridge.

12. The system of claim 11, wherein the at least one strengthening ridge comprises a ridge running around at least a portion of the perimeter of the reflective paddle.

13. The system of claim 8, wherein the plurality of MSSS includes a micromembrane.

14. The system of claim 1, wherein the property of the reflected beam of light is a position.

15. The system of claim 1, wherein the property of the reflected beam of light is an intensity of at least a portion of an interference pattern.

16. The system of claim 1, further including one or more reference sensors.

17. The system of claim 1, further comprising:
a sensor base portion including a plurality of reservoirs, wherein various sensors of the plurality of sensors are disposed in different reservoirs.

18. The system of claim 17, wherein a portion of the plurality of sensors are disposed in one reservoir.

19. The system of claim 16, further comprising:
a sensor base portion including a plurality of reservoirs, wherein the at least one reference sensor is disposed in a different reservoir from the sensors including the associated probe molecules.

20. The system of claim 17, wherein the sensor base portion further includes a plurality of access channels, wherein each of the plurality of access chambers are associated with one of the plurality of reservoirs.

21. A system for detecting target molecules, comprising:
a pair of sensors, including a functionalized sensor with associated probe molecules for interacting with the associated target molecules, the functionalized sensor exhibiting a physical change upon interacting with the associated target molecules, and a non-functionalized reference sensor; and
an optical detector, the optical detector positioned to receive a reflected beam of light from each of the sensors, wherein a property of the reflected beam of light for the functionalized sensor changes when the associated probe molecule interacts with the associated target molecules.

22. The system of claim 21, wherein each of the sensors are microcantilevers.

23. A method, comprising:
functionalizing a plurality of sensors, wherein each of the plurality of sensors is functionalized to detect a different target material;
introducing sample fluids or gasses into regions proximate to each of the plurality of sensors;
reflecting light from a surface of each of the plurality of sensors; and
detecting reflected light from each of the plurality of sensors.

24. The method of claim 23, wherein the target material comprises at least one of the group consisting of:
a short single-stranded DNA sequence, an antigen, and a protein.

25. The method of claim 23, further including reflecting light from the surface of at least one reference sensor.

26. The method of claim 25, further including detecting reflected light from the at least one reference sensor.

27. The method of claim 23, wherein the plurality of sensors includes a first sensor including an associated probe molecule, and wherein the method further includes determining whether a sample fluid or gas for the first sensor includes a target material associated with a probe molecule.

28. The method of claim 27, wherein the determining includes comparing the position of the reflected light from at least one of the plurality of sensors at a first time with the position of the reflected light from the at least one of the plurality of sensors at a second time.

29. The method of claim 23, wherein introducing sample fluids or gasses into regions proximate to each of the plurality of sensors comprises introducing sample fluids or gasses into a plurality of individual reservoirs dedicated to individual sensors.

30. The method of claim 23, wherein introducing sample fluids or gasses into regions proximate to each of the plurality of sensors comprises introducing sample fluids or gasses into a plurality of individual reservoirs dedicated to individual groups of sensors.

31. The method of claim 23, wherein detecting the reflected light from each of the plurality of sensors comprises:
determining the position of the reflected light by calculating a spatial average of the intensity distribution of a spot of the reflected light.

32. The method of claim 31, further comprising:
rejecting individual pixel values from the calculation when the intensity of the pixels fall below a threshold value.

33. The method of claim 31, further comprising:
applying a gain factor algorithm to the calculation to give more weight to pixels having high sensitivity and low noise.

34. The method of claim 31, further comprising:
compensating for sensor drift over time.

35. The method of claim 26, wherein the plurality of functionalized sensors includes a first sensor including an associated probe molecule, and wherein the method further includes determining whether a sample fluid or gas for the first sensor includes a target material associated with a probe molecule by comparing the position of the reflected light from the first sensor with the position of the reflected light from the at least one reference sensor over time.

36. The method of claim 23, wherein each of the functionalized sensors are positioned in individual dedicated reservoirs.

37. The method of claim 23, wherein some of the plurality of functionalized sensors are positioned in the same reservoir.

38. The method of claim 26, wherein the reference sensor is positioned in a different reservoir from the plurality of non-reference sensors.

39. A method, comprising:
functionalizing a plurality of sensors, wherein each of the plurality of sensors is functionalized to detect the same target material;
introducing sample fluids or gasses into regions proximate to each of the plurality of sensors;
reflecting light from a surface of each of the plurality of sensors; and
detecting reflected light from each of the plurality of sensors.

40. The method of claim 39, further comprising:
wherein the plurality of functionalized sensors each include the same associated probe molecule, and wherein the method further includes determining whether a sample fluid or gas includes the target material by comparing the position of the reflected light from the plurality of functionalized sensors with the position of the reflected light from the at least one reference sensor over time.

41. A system, comprising:
a sensor base portion, the sensor base portion including a plurality of separate regions for holding fluid or gas, the sensor base portion further including a plurality of access channels, each of the plurality of access channels associated with one of the plurality of regions; and
a plurality of sensors, each of the plurality of sensors formed proximate to an associated one of the plurality of regions.

42. The system of claim 41, wherein the sensor base portion comprises silicon.

43. The system of claim 42, wherein the sensor base portion further comprises glass.

44. The system of claim 41, wherein the plurality of sensors comprises a plurality of MSSS.

45. The system of claim 44, wherein the plurality of sensors comprise microcantilevers.

46. The system of claim 45, wherein the microcantilever comprises a reflective paddle portion.

47. The system of claim 46, wherein the reflective paddle portion comprises at least one strengthening ridge.

48. The system of claim 41, further including at a reference sensor.

49. The system of claim 41, wherein each of the plurality of access channels has an inlet for providing a functionalization fluid or gas.

50. The system of claim 49, wherein each of the plurality of access channels has an outlet.

51. The system of claim 49, wherein the plurality of access channels share a common outlet.

52. The system of claim 51, wherein the common outlet is to provide a rinsing fluid or gas to each of the plurality of access channels.

53. The system of claim 51, wherein the common outlet is to provide a sample fluid or gas to each of the plurality of access channels.

54. The system of claim 49, wherein the inlet is configured to receive a micropipette for providing the functionalization fluid or gas.

55. The system of claim 41, further comprising:
an optical detector, the optical detector positioned to receive a reflected beam of light from each of the plurality of sensors, wherein a property of the reflected beam of light for each of the plurality of sensors changes when the associated probe molecule interacts with the associated target molecules.

56. The system of claim 41, wherein the each one of the plurality of sensors are disposed in a different separate region for holding fluid or gas.

57. The system of claim 41, wherein a portion of the plurality of sensors are disposed in one of the regions for holding fluid or gas.

58. The system of claim 41, wherein each of the plurality of sensors are positioned to be surrounded by fluid of gas when each of the separate regions for holding fluid or gas are filled with fluid or gas.

59. A microcantilever for use in a system for detecting target molecules, comprising:
a microcantilever body;
one or more associated probe molecules on the microcantilever body for interacting with the associated target molecules; and
a reflective paddle portion.

60. The system of claim 59, wherein the reflective paddle portion comprises at least one strengthening ridge.

61. The system of claim 60, wherein the at least one strengthening ridge comprises a ridge running around at least a portion of the perimeter of the reflective paddle.
